(19) United States
(12) Patent Application Publication
(10) Pub. No.: US 2019/0134464 A1
(43) Pub. Date: May 9, 2019

(54) EXERCISE TRAINING SYSTEM AND METHOD

(71) Applicant: Paul ANDERTON, Fairview Park (AU)

(72) Inventor: Paul ANDERTON, Fairview Park (AU)

(21) Appl. No.: 15/971,382

(22) Filed: May 4, 2018

Related U.S. Application Data
(63) Continuation of application No. 14/404,797, filed on Dec. 1, 2014, now abandoned.

(30) Foreign Application Priority Data
May 30, 2012 (AU) 2012902248

(51) Int. Cl. A63B 24/00; G09B 19/00; A61B 5/103

(52) U.S. Cl. CPC A63B 24/0062; A63B 2024/0065; A61B 5/103; G09B 19/0038

(57) ABSTRACT

A method of analysing a resistance exercise activity executed by a subject is disclosed. In an embodiment, the method includes:
receiving a plurality of exercise parameter values for the executed exercise activity into a processing device, the plurality of exercise parameter values including information representing an execution profile (EP) for the executed exercise activity;
accessing a store of information to retrieve information representing a load profile (LP) for the executed exercise activity; and
processing the plurality of exercise parameter values and the information representing the load profile to determine one or more assessment parameter values for assessing the executed exercise activity.

This is a continuation application of U.S. application Ser. No. 14/404,797, filed Dec. 1, 2014, which is a national stage entry of PCT/AU2013/000571, filed May 30, 2013, which claims priority to AU 2012902248, filed May 30, 2012. The disclosures of each reference are hereby incorporated by reference in their entireties.

### FIELD OF THE INVENTION

The present invention relates to exercise training for skeletal muscle growth. In a typical application an embodiment of the present invention may find application in an exercise training programme, such as a resistance training program.

### BACKGROUND OF THE INVENTION

Resistance training is the practice of placing a subject's skeletal muscles under load, typically via eccentric and concentric contractions of a prescribed movement for a number of consecutive repetitions (“reps”) which may be repeated several times (“sets”) with a rest between each set. In some forms of resistance training, the aim of the training is to elicit an increase in muscle size or strength or both.

Generally speaking, conventional training methods employ variations of the set/repetition schemes and/or variations of contraction profiles (exercise execution) to stimulate (stress) a muscle or muscles and generate a response in the muscle or muscles worked by the exercise, with some methods being more effective than others and different for each individual due to genetic/physiological differences. For example, a set/rep type exercise may be designed to create a training response which is biased towards a particular outcome, such as heavy weights with, for example, 4 reps or less for predominantly strength gains and, for example, 8 to 12 reps for size gains (hypertrophy) of a muscle group worked by the exercise.

Furthermore, within these conventional training methods there are other variables which may be varied for the performance of the ‘exercises’, including but not limited to:
the speed of the repetition, both in a concentric phase and an eccentric phase of a muscle activation and the ‘pause’ between both;
the ‘tempo’ of the repetition, being the combination of all phases;
the load profile, which may be considered as the “effective” load on the working muscles at each stage of the exercise as the muscles contract and the forces vary due to, for example, a changing angle of incidence of gravity in relation to the skeletal structure, changing factors of leverage of the joint(s) and tendon(s) involved, or the load profile of, for example, a camshaft if an exercise machine incorporating a cable and/or camshaft is used, or simply the technique of the exercise performance such that the trainee deliberately alters their stance, position, or limbs during the exercise to elicit a particular load effect; and
various ‘overload’ techniques designed to increase the overall stress on the muscle—including but not limited to: forced reps, rest-pause, pre-exhaust exercises, drop sets, compound sets, giant sets, and “x” reps.

A fundamental consideration when preparing a training programme incorporating exercise activities is that every person is different. Also, for each person, the various muscles will be different in their ability to handle and recover from stresses. In this respect, muscles are recognised as including various fibre “types”, broadly classified as “fast-twitch” and “slow-twitch” fibres, and also as having have several sub-categories. Each fibre “type” is recognised as having abilities biased towards particular training loads. For example, slow-twitch fibres are recognised as being better in endurance events, whereas fast-twitch fibres are recognised as being better at short term explosive or power events.

It is also known that the different fibre types will come into play at various stages of physical performance based upon the loads and duration of an exercise or physical activity.

Due to physiological differences between trainees, such as those outlined above, the variable options of training schemes, the time it takes to notice results, and various other factors which may impact on the training response of a trainee during training (such as, nutritional variations, sleep patterns, psychological stresses and the like), there is often significant confusion as to how to achieve a desired training effect, and thus which type of training approach to adopt. Consequently, trainees may employ a protocol (such as a training program) that is not optimised for their physiology, or at least not tailored for, their desired objectives, or other circumstances. As an example, a trainee may adopt a training program from a sports person they admire, unaware of, or ignoring the fact that, that sports person has different genetic structure, or lives a different lifestyle, amongst other things.

Another difficulty with existing “generic” training protocols is that they may have prescribed set, rep, and recovery schemes which ignore the physiological differences between people. Because of this, any “fixed” training “scheme” may only be suitable for a small percentage of the population, and indeed only suited for a period of time until the trainee adapts to that scheme, or reaches a different “stage” in their life either due to their training progression, age, health, or living conditions.

### SUMMARY OF THE INVENTION

A first aspect of the present invention provides a method of analysing a resistance exercise activity executed by a subject, the method including:
a) receiving a plurality of exercise parameter values for the executed exercise activity into a processing device, the plurality of exercise parameter values including information representing an execution profile (EP) for the executed exercise activity;
b) accessing a store of information to retrieve information representing a load profile (LP) for the executed exercise activity; and
c) processing the plurality of exercise parameter values and the information representing the load profile to determine one or more assessment parameter values for assessing the executed exercise activity.

Examples of resistance training exercise include strength training exercises such as isotonic and/or isometric exercises. The exercise activity may involve exercise equipment such as resistance bands, free weights, or exercise machines. Examples of isotonic exercises include squats, bench press, lat pull downs, dumbbell flyes, cable flyes, bar bell curls, calf raises, chin ups, sit ups, push-ups, and the like.

The exercise activity may involve one or more weights (wt), one or more sets (s) including one or more repetitions (R), and a total activity (elapsed) time (Tₑ) for the exercise. In an embodiment, the plurality of exercise parameters include:
a) a set parameter value (n) representing the number of the one or more sets for the resistance training activity;
b) a weight parameter value (wtₛ) for the weight associated with each repetition within a set (s);
c) a repetition parameter value (Rₛ) representing the number of one or more repetitions in a s where s=1 to n; and
d) a total activity (elapsed) time parameter value (Tₑ).

In one embodiment, the execution profile information and the load profile information include corresponding sequences of values, wherein each value represents a duration of a different exercise phase of the exercise activity. The values representing the duration of different exercise phases of the exercise activity may include values representing the duration of:
a) an eccentric phase;
b) an eccentric-pause phase;
c) a concentric phase; and
d) a concentric-pause phase.

The load profile (LP) information may be expressed as:

LP=[d₁,d₂,d₃,d₄]
where:
   d₁=value indicating whether the eccentric phase is intended to contribute to stress;
   d₂=value indicating whether the eccentric-pause phase is intended to contribute to stress;
   d₃=value indicating whether the concentric phase is intended to contribute to stress; and
   d₄=value indicating whether the concentric-pause phase is intended to contribute to stress.

In an embodiment, the execution profile (EP) information is expressed as:

EP=[t₁,t₂,t₃,t₄]
where:
   t₁=the duration of the eccentric phase;
   t₂=the duration of the eccentric-pause phase;
   t₃=the duration of the concentric phase; and
   t₄=the duration of the concentric-pause phase.

Processing the plurality of exercise parameters values and the activity information to determine one or more assessment parameter values for assessing the executed exercise activity may include multiplying corresponding values of the actual execution information and the load profile information to obtain a sequence of products, and summing the products to obtain a single value indicative of a working time under tension (TUT) for a muscle targeted or intended to be activated by the exercise activity. For example, wherein for each repetition (r) within a set (s), the single value indicative of a working time under tension (TUT) may be determined as:

TUTᵣ=t₁·d₁+t₂·d₂+t₃·d₃+t₄·d₄

or more generally expressed for a set (s) as the average total time under tension (TUT_{R}) per repetition within the set (s) including a number of repetitions (Rₛ) as:

$${TUT}_{R} = \frac{{\sum }_{r = 1}^{{R}_{s}} {t}_{1 , r} \cdot {d}_{1 , r} + {t}_{2 , r} \cdot {d}_{2 , r} + {t}_{3 , r} \cdot {d}_{3 , r} + {t}_{4 , r} \cdot {d}_{4 , r}}{{R}_{s}}$$

in cases where each repetition in a set (s) involves the same load profile and execution profile, and each set includes a number (Rₛ) of repetitions.

It is preferred that the one or more assessment parameter values for assessing the executed exercise activity include:
a) a work volume parameter value for the executed exercise activity (W);
b) a work intensity parameter value for the executed exercise activity (Wᵢ);
c) a stress intensity parameter value for the executed exercise activity (Sᵢ); and
d) a hypertrophy factor parameter value for the executed exercise activity (H_{f}).

In an embodiment, the work volume parameter value (W) may be determined as:

W=Σₛ₌₁ⁿWₛ=Σₛ₌₁ⁿ(Rₛ·wtₛ)
where:
   n is the number of sets in the exercise activity;
   Rₛ is the number of reps in set s, where s=1 to n; and
   wtₛ is the weight associated with the set where s=1 to n.

The work intensity parameter value (Wᵢ) may be determined as:

$${W}_{i} = \frac{W}{{T}_{e}}$$

Where Te is the elapsed time of the exercise activity.

The stress intensity parameter value (Sᵢ) may be determined as:

$${S}_{i} = \frac{{\sum }_{s = 1}^{n} {R}_{s} \cdot {wt}_{s} \cdot {TUT}_{R}}{{T}_{e}}$$

The hypertrophy factor parameter value (H_{f}) may be determined as:

H_{f}=Sᵢ·W

Some embodiments of the present invention thus relate to a system and method which assesses a plurality of exercise parameters relating to resistance training, including a “hypertrophy factor” (H_{f}) that is proportional to stress intensity and work capacity of a muscle group(s), to guide the subject to set a tailored target for improved training results.

A second aspect of the present invention provides a computer readable media including computer program instructions which are executable by a processor to implement a method according to the first aspect of the present invention.

In a third aspect of the present invention there is provided a system for analysing a resistance exercise activity executed by a subject, the system including:
a) a processing unit programmed with a set of program instructions in the form of a computer software program;
b) a store of information representing a load profile (LP) for the executed exercise activity; and
c) means for receiving a plurality of exercise parameter values for the executed exercise activity into a processing device, the plurality of exercise parameter values including information representing an execution profile (EP) for the executed exercise activity;
   wherein the processing unit retrieves the information representing the load profile (LP) for the executed exercise activity, and processes the plurality of exercise parameter values and the information representing the load profile to determine one or more assessment parameter values for assessing the executed exercise activity.

In a fourth aspect of the present invention there is provided a method of determining plural sets of instructions including one or more exercise parameters for execution of a resistance exercise activity by a subject, the method including:
accessing a store of information to retrieve a set of parameter values attributable to the subject's prior performance of the exercise activity;
processing a user-selected adjustment of a target criteria for the exercise activity and the retrieved set of parameter values to determine the at least one set of instructions including the one or more exercise parameters for executing an exercise activity to be selected by the user; and
outputting the at least one set of instructions for the selected exercise activity.

According to a fifth aspect of the present invention there is provided a system for determining one or more exercise parameters for execution of a resistance exercise activity by a subject, the system including:
a) a processing unit programmed with a set of program instructions in the form of a computer software program;
b) a store of information in the form of a set of parameter values attributable to the subject's prior performance of the exercise activity; and
c) wherein the computer software program is executable by the processor to cause the processor to:
   obtain the subject's target criteria for one or more exercises objectives;
   retrieve from the store of information a set of parameter values attributable to the subject's prior performance of the exercise activity;
   process the target criteria and the set of parameter values to determine at least one set of instructions including the one or more exercise parameters for executing the exercise activity; and
   output the at least one set of instructions.

Yet another aspect of the present invention provides a method of analysing a resistance exercise activity executed by a subject, the method including:
receiving a plurality of exercise parameter values for the executed exercise activity into a processing device, the plurality of exercise parameter values including information representing an execution profile (EP) for the executed exercise activity;
accessing a store of information to retrieve information representing a load profile (LP) for the executed exercise activity; and
processing the plurality of exercise parameter values and the information representing the load profile to determine one or more assessment parameter values for assessing the executed exercise activity;
wherein the execution profile information and the load profile information include corresponding sequences of values associated with a different exercise phase of the exercise activity for a muscle of the subject intended to perform work during the exercise activity, such that the values associated with different exercise phases of the exercise activity include values associated with:
   an eccentric phase;
   an eccentric-pause phase;
   an concentric phase; and
   a concentric-pause phase;
and wherein the load profile (LP) information identifies the exercise phases intended to contribute to work during execution of the exercise activity, and the execution profile (EP) information identifies the duration of each exercise phase which contributed to work during execution of the exercise activity

Embodiments of the present invention may provide a method of analysis and/or predictive modeling of resistance training parameters which provides a tool which may be used to customise exercise activities for an individual based on their individual physiological characteristics and training response as determined from past performance. For example, embodiments of the present invention may provide analysis of, and predictive modelling for, a subject (such as an individual trainee) to firstly determine training parameters for achieving their training goals via iterative analysis and guidance. It is further possible that embodiments may provide feedback to adjust a program of exercise activity to maintain progress towards the subject's training objectives irrespective of potential changes in the trainee's physiology or other variables that can affect the training response.

Embodiments of the present invention may thus assist in overcoming obstacles with regard to ‘knowing’ a plurality of factors that contribute to athletic performance for example, a trainee's muscle make-up (via biopsies) and recuperative abilities, energy systems, oxygen delivery, hormonal responses, (i.e. ‘the system’ characteristics) and the subsequent assumptions of the ‘optimal’ training that may therefore result, by assessing the actual performance of the total ‘system’ and providing feedback and guidance to ‘peak’ training parameters via predictive modelling.

Embodiments of the present invention may provide a means of analysing performance of an exercise activity via exercise parameters which may be recorded during execution of an exercise activity without requiring complex technical equipment or measurement instruments. Embodiments of the present invention may thus be simple to implement in a training environment without requiring any modifications to the existing training equipment, such as gym equipment.

Furthermore, by monitoring training outputs (performance statistics outlined earlier) against training input (for example, weight, repetitions, number of sets, load profile, time parameters outlined earlier) embodiments of the present invention may tailor or at least customise to some extent the training stimulus for a trainee.

This approach adopted by the present invention is expected to provide improved accuracy compared to technical equipment or measurement instruments which focus on one or more factors of performance (for example, muscle fibre type) and which do not take into account other factors of performance (for example, energy systems or oxygen delivery) that may ultimately affect the actual stress that can be imposed on the muscle and thus may result on a lower stress to the muscle than determined by the assumptions. This problem is particularly evident with forms of resistance training which work on the principle of knowing or assuming one or more parameters and which then make assumptions in relation to other parameters based on generic paradigms of a person's physiology.

The present invention may involve determining the optimal peak stress and workload and “hypertrophy factor” by monitoring and analysing exercise parameters and analysing parameters to provide performance statistics as well as predictive training parameters and targets to accelerate progress towards the subject's training goals.

Embodiments of the present invention may relate to an automated system for resistance training which thus provides tailored training protocols for each individual trainee to guide them to their optimal training parameters for each muscle and exercise. It is preferred that the system uses analysis of training parameters that may be measured without specialised equipment and uses analysis and predictive modeling to iteratively determine the optimal training mode for each exercise and/or muscle for each trainee.

Some embodiments of the present invention may address shortcomings with training protocols which rely on prescribed set, repetition, and recovery schemes and ignore the physiological differences between people. Such training protocols may only be suitable for a small percentage of people whose physiology suits those protocols and, even then, for a period of time until a trainee adapts to that protocol or perhaps reaches a different “stage” in their life either due to their age, health, living conditions, or other circumstances.

An advantage of the present invention is that it may provide an assessment and iterative process which is capable of tailoring exercise activities for the subject.

### BRIEF DESCRIPTION OF THE DRAWINGS

An illustrative embodiment of the present invention will be discussed with reference to the accompanying drawings wherein:

FIG. 1 is a block diagram of a system according to an embodiment of the present invention;

FIG. 2 is a flow diagram for a method according to an embodiment of the present invention;

FIG. 3 is a table including load profile and execution profile information for an exercise activity;

FIG. 4 is a table including load profile and execution profile information for a plurality of exercise activities;

FIG. 5 is a diagram illustrating example bio-mechanical processes for executing a bicep curl exercise activity to illustrate the effects on exercise performance on the load profile;

FIG. 6 is a table including load profile and weight information for a plurality of exercise activities;

FIG. 7 is a table listing weight and repetition information with reference to a one-repetition maximum value for an exercise activity; and

FIG. 8 is an example output report including plural instructions for executing an exercise activity.

### DESCRIPTION OF PREFERRED EMBODIMENT

A detailed description of one or more preferred embodiments of the invention is provided below along with accompanying figures that illustrate by way of example the principles of the invention. While the invention is described in connection with such embodiments, it should be understood that the invention is not limited to any embodiment. On the contrary, the scope of the invention is limited only by the appended claims and the invention encompasses numerous alternatives, modifications, and equivalents. For the purpose of example, numerous specific details are set forth in the following description in order to provide a thorough understanding of the present invention. The present invention may be practiced according to the claims without some or all of these specific details. For the purpose of clarity, technical material that is known in the technical fields related to the invention has not been described in detail so that the present invention is not unnecessarily obscured.

The steps of a method or algorithm described in connection with the embodiments disclosed herein may be embodied directly in hardware, in a software module executed by a processor, or in a combination of the two. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. Software modules, also known as computer programs, computer codes, or instructions, may contain a number of source code or object code segments or instructions, and may reside in any computer readable medium such as a RAM memory, flash memory, ROM memory, EPROM memory, registers, hard disk, a removable disk, a CD-ROM, a DVD-ROM or any other form of computer readable medium. In the alternative, the computer readable medium may be integral to the processor. The processor and the computer readable medium may reside in an ASIC or related device. The software codes may be stored in a memory unit and executed by a processor. The memory unit may be implemented within the processor or external to the processor, in which case it can be communicatively coupled to the processor via various means as is known in the art.

The term “software,” as used here in, includes but is not limited to one or more computer readable and/or executable instructions that cause a computer or other electronic device to perform functions, actions, and/or behave in a desired manner. The instructions may be embodied in various forms such as routines, algorithms, modules or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skilled in the art that the form of software is dependent on, for example, requirements of a desired application, the environment it runs on, and/or the desires of a designer/programmer or the like.

Embodiments of the present invention apply analysis to exercise parameters recorded during or after execution of an exercise activity to determine assessment parameters for providing feedback to the trainee (hereinafter, the “subject”) of the actual training statistics as well as a predictive target of exercise parameters that will modify the parameters according to the subject's abilities. Although the following description relates to an implementation of an embodiment of the invention for use by the subject, it will be appreciated of course that the present invention could be implemented for use by a trainer, coach, or the like. Indeed, it is possible that embodiments of the present invention could be implemented on-line (suitable, for example, for internet based remote coaching) or as a mobile application.

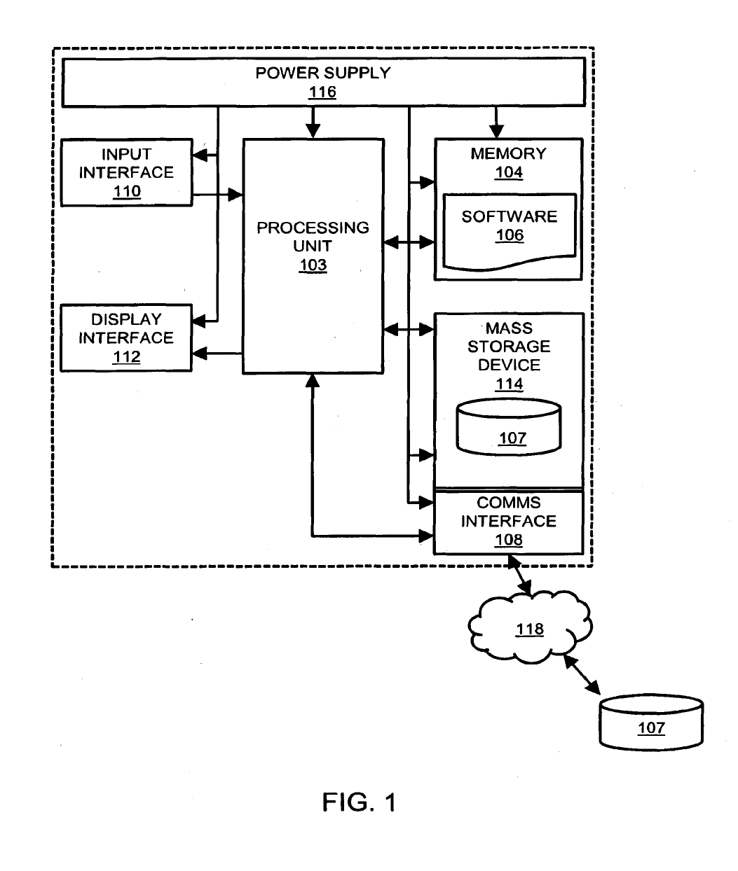

Turning initially to FIG. 1 there is shown a block diagram for a system 100 according to an embodiment of the present invention. The system 100 includes a processing device 102 such as a desktop computer, a lap top computer, a note book computer, a hand held computer, a programmed electronic device equipped with a programmed or programmable controller (such as a microcontroller), a smart phone, a personal digital assistant or the like. It will thus be understood that the term “processing device” is intended to denote any type of device including a processor capable of executing a set of software instructions to perform a function. The processing device 102 includes a processing unit 103, a memory 104, a computer software program 106 resident in a first store of information in the form of a memory 104, communications interface 108, input interface 110, display interface 112, mass storage device 114, and power supply 116, however, it will be appreciated that any configuration is acceptable such that the functionality of accepting data inputs, processing data and providing outputs in any format can be accommodated, either within a hardware device or any “virtual” or “cloud” device or process-capable medium, hereinafter encapsulated by the term “processing device” or “device 102”. Hereinafter, all references to specific elements of the device are taken to also address any other configurations of the processing system.

The memory 104 may be installed on-board the processing device 102 or may be connectable or accessible to the processing device 102 via a suitable communications interface, such as communications interface 108. A suitable communications interface includes a universal serial bus (USB) interface. The memory 104 may include volatile (for example, RAM, DRAM, SRAM) or non-volatile memory, such as a ROM memory (for example, PROM, EPROM, EEPROM), or NVRAM (such as FLASH) or the like. One example of a suitable memory is a USB FLASH memory adapted to communicate with the processing unit 103 via a USB interface.

The memory 104 is programmed with a set of executable instructions in the form of the computer software program 106. For the purpose of this description, references to the term “memory” are to be understood to denote the total memory available to the processing unit.

The software 106 will also include an operating system for controlling system functions. Suitable operating systems will depend on the processing unit 103 and would be well known to a skilled addressee.

In addition to storing the executable instructions, the memory 104 may also store a database 107. Alternatively, the database 107 may be stored on a remote device, such as a server, or a device which is accessible to the processing device 102 via the or another communications interface 108 which may support wired or wireless communications with a network 118, such as the internet.

The communications interface 108 may thus include a wired interface such as a USB, Ethernet or the like, or alternatively may include a wireless interface such as a Wi-Fi interface, a Bluetooth interface, or the like. Other suitable communications interfaces would be known to a skilled addressee.

The communications interface 108 may support data communication which allows the database 107 to be accessible to the processing device 102 via a cloud. In this respect, the database 107 may include multiple databases distributed across a plurality of network accessible devices.

The database 107 stores information for one or more exercise activities, such as weight training type exercises. The stored information includes load profile information (LP) for one or more exercise activities as the approximate “load” of the weight on the working muscle(s) (that is, the muscle intended to be exercised by the exercise activity) at four distinct phases of the exercise, expressed as components |Eccentric |Pause |Concentric |Pause | where the elements are either 1 or 0 (that is, 1111, or 1011, or 1011). The load profile will be described in more detail following. In some embodiments, the database 107 also stores additional information for the one or more exercise activities such as the weight component lifted as a ratio of the weight loaded on the machine or bar (for example, 10%, 50%, 100%, 150%) as a result of leverage, cams, or angle of lift versus angle of incidence with gravity, the inherent and “minimum” weight as a result of the weight of the machine (for example, the foot plate of a leg press machine), and the component of bodyweight lifted in the exercise activity (for example, 10%, 50%, 100%, 150%). In the case of squats where part of the body (lower legs) are supported by the floor and not part of the lifting load on the “working” muscles, or in the case of chinning where the arms are supported by the chinning bar and the weight lifted is therefore the remainder of the body and any weight attached via a weight belt or otherwise held by the subject. Any variations of the above may apply and in some cases, multiple components will apply (for example, inclined hack squats where there is a minimum weight of the machine (the slide and shoulder pad supports), a percentage of the bodyweight lifted as with squats, and the addition of these two weights is effectively reduced by the angle of the machine such that it is not directly aligned with the force of gravity. Thus, for example, the total weight (wt) may be expressed as:

{[machine slide weight]+[added weight]+% bodyweight}×[% Angle gravity component]

Furthermore, the database 107 also stores additional information in the form of a repository of historical training (exercise activities) records for exercise activities which have been executed by a subject to enable recall and analysis either for comparison purposes or for processing by the processing device 102 to generate a set of training instructions for executing the exercise activity based on prior execution information.

Referring again to FIG. 1, the display interface 112 may include a conventional display screen, such as an LCD display but may also be any form of display medium including hologram or “virtual” screens. The input interface 110 may include a keyboard, track-ball, touch-screen, mouse pointer, keypad, audio input, or “virtual” position sensor or the like. Suitable input interface devices would be well known to a skilled addressee.

In the present case, the input interface 110 provides a receiving means for receiving a plurality of exercise parameter values for an executed exercise activity into the processing device 102, and/or a means for providing output information. For example, in some embodiments the user interface 110 may provide:
input fields to record required data to analyse executed exercise activities; and
an output display (such as a “dashboard”) of the exercise activities executed to date and mechanism for prescribing new targets for future exercise activities.

The input interface 110 may include an interactive graphical user interface (GUI) which permits a subject to, for example, enter and select parameter values for an exercise activity. Other suitable input options may include sensor devices (for example, accelerometer(s), or strain gauges) adapted with communication means, or specifically built training apparatus (with in-built detectors and data transmission systems) which are adapted to communicate data to an input device such as a smart phone, tablet, laptop computer, desktop computer, wrist watch based device, or other intelligent device, via a suitable wired or wireless interface. Such sensor devices may include devices which are worn by the subject during the exercise activity. The sensor devices may communicate with an input device via a suitable wired or wireless interface, or may feed data directly to a communications network or to a processing device locally or via the cloud.

The plurality of exercise parameter values will include information representing an execution profile (EP) for an executed exercise activity.

The processing unit 103 processes a plurality of received exercise parameters values for an executed exercise activity, including information representing the Load profile (LP), and possibly other information, to determine one or more assessment parameter values for assessing the executed exercise activity. The processing unit 103 then retrieves from the database 107, information representing the exercise parameters, which may include the ratio of weight lifted, % bodyweight lifted, inherent machine weight, load profile (LP) for the executed exercise activity, and processes the plurality of exercise parameters values including the execution profile (EP) and the information representing the load profile (LP) to determine one or more assessment parameter values for assessing the executed exercise activity.

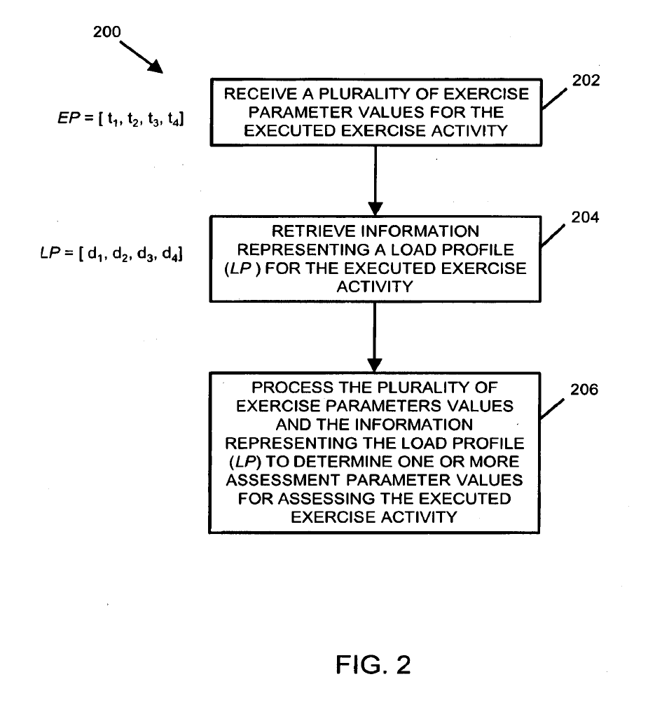

FIG. 2 shows a flow diagram for a method according to an embodiment of the present invention. As shown, the illustrated method involves receiving, at step 202, a plurality of received exercise parameter values for the executed exercise activity, wherein the information for the executed exercise activity includes information representing the execution profile (EP).

In embodiments in which the exercise activity executed by a subject includes a resistance training exercise activity involving one or more weights (wt), one or more of sets (s) including one or more repetitions (R), and a total activity time (Tₑ), the plurality of exercise parameter values will also include:
a) a set parameter value (n) representing the number of the one or more sets (s) for the resistance training activity;
b) a weight parameter value (wtₛ) for the weight associated with each rep within a set (s) where s=1 to n;
c) a repetition parameter value (Rₛ) representing the number of one or more repetitions in a s where s=1 to n; and
d) a total activity time parameter value (Tₑ).

After the exercise parameter values for the executed exercise activity have been received into the processing device 102, information representing the load profile for the executed activity is then retrieved, at step 204, from the database 107. The plurality of exercise parameters values and the information representing the load profile is then processed, at step 206, to determine one or more assessment parameter values for assessing the executed exercise activity.

The step of processing a plurality of exercise parameters values and the information representing the load profile will now be explained in more detail.

As explained above, the load profile information includes information which identifies the phases of the exercise activity during which a muscle targeted by the exercise activity is intended to perform work, and/or contribute to stress on the muscle. On the other hand, the execution profile information includes information which relates to the duration of each phases of the exercise activity, as measured during execution of the exercise activity, irrespective of which phase was intended to contribute to stress for that exercise activity.

In this respect, a resistance training type exercise activity involving manipulating (such as lifting and lowering) a weight (wt) for a set including a number of repetitions includes four phases of a repetition, namely an eccentric phase, an eccentric-pause phase, a concentric phase, and a concentric-pause phase, with each phase having an associated duration. In an embodiment of the present invention, the load profile may be represented as a binary digit code or “mask”, including values which identify the phases of an exercise activity that are intended to contribute to stress for that exercise activity. For example, the load profile (LP) may be expressed in a general form as:

LP=[d₁,d₂,d₃,d₄]

where:
d₁=value for indicating whether the eccentric phase is intended to contribute to stress
d₂=value for indicating whether the eccentric-pause phase is intended to contribute to stress
d₃=value for indicating whether the concentric phase is intended to contribute to stress
d₄=value for indicating whether the concentric-pause phase is intended to contribute to stress

It will of course be appreciated that the load profile (LP) may be expressed in a different form.

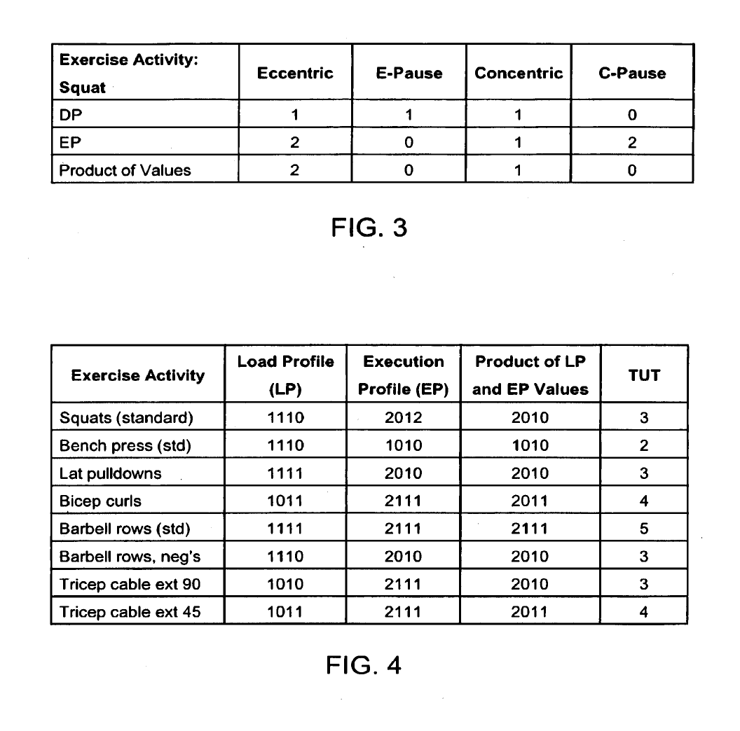

With reference now to an example shown in FIG. 3, the load profile for a “squat” requiring an eccentric phase duration, an eccentric-pause duration, a concentric phase duration, and no concentric-pause phase duration the LP may be expressed, using the above-described general form, as:

LP=1 1 1 0

The above expression applies if the exercise activity is performed such that the knees are locked at the top of the movement and the load is effectively removed from the working muscles (primarily quadriceps)—which may be referred to as “lockout squats” in the database for example. However, if the exercise activity is performed such that the knees are not locked and the load is always present on the quadriceps, the exercise may be referred to as “non-lockout squats”, in which case the load profile may be expressed, using the above-described general form, as:

LP=1 1 1 1

In an embodiment of the present invention, the subject undertaking the exercise activity may be able to select the appropriate LP expression for the exercise by selection of the respective exercise activity from the database.

In an embodiment, the execution profile information may represent a sequence of values, wherein each value represents a duration of one of the exercise phases of the exercise activity. For example, the execution profile (EP) may be expressed in a general form as:

EP=[t₁,t₂,t₃,t₄]

where:
t₁=the duration of the eccentric phase
t₂=the duration of the eccentric pause phase
t₃=the duration of the concentric phase
t₄=the duration of the concentric-pause phase

In practice, the duration values t₁, t₂, t₃, and t₄ may be recorded during execution of an exercise activity using an suitable means, such as a stop watch. Verification to check approximate accuracy of the estimate may be achieved by the easier method of timing a set (for example, if the estimated/targeted profile is 1110 and each repetition is 10 seconds in duration, the total duration of the set is 30 seconds). The duration values may then be input into the system at the completion of the exercise activity for analysis by the system.

It is possible that in some embodiments, the duration values t₁, t₂, t₃, and t₄ may be automatically recorded by a sensing device worn by the subject or attached to a machine, or incorporated in an exercise apparatus being operated by the subject. Such a sensing device may include, for example, a wireless sensor. Examples of a wireless sensor include a band or strap incorporating a sensor which is worn on, for example, the wrist of the subject to detect movements during exercise activities such as a bench press, pull-downs, bicep curls, tricep extensions. In another example, the sensing device may be worn as a waist band, or as a necklet during chin-ups, or relocated to the ankle during leg curls or leg extensions.

In other words, a sensing device would preferably be placed on a part or parts of the body involved with a primary range of movement targeted by the exercise activity, for example, by placing the sensing device on the waist (or worn on the torso) during chin-ups as opposed to the wrist, since the wrist does not move during chin-ups whereas the waist or torso area is involved with the primary or target range of motion used to stimulate the working muscles (latissimus dorsi). Similarly, the sensing device could remain on the wrist during squats, for example, since the wrist typically remains on the barbell at neck level and is therefore subject to the full range of up/down motion during the exercise activity. If the subject was performing leg presses however, the strap would be relocated to the ankle or the weight plate, or in the case of a cable-weight machine, the device could be attached to the weight stack. Indeed, in most instances where a cable-weight stack is used, the weight stack may provide a preferable location for the sending device regardless of the body movements, as the weight stack is subject to the true range of motion.

In some embodiments of the present invention, the activity database for the system may include reference data for interpreting sensor data obtained from a sensing device attached to the weight stack, including:
a) direction of movement of the weight stack that corresponds to the subject's concentric/eccentric movements; and
b) adjustment of the actual weight borne by the subject according to, for example, a pulley configuration of the machine, i.e. a single pulley means a 1:1 ratio of the weight on the stack and the weight lifted, whereas a two-pulley arrangement generally means that the weight lifted is half that on the stack etc.

Embodiments of the present invention preferably access an activity database which is indexed to identify the above criteria, and for the subject and/or coach to make adjustments in the interpretation of sensor data if necessary.

As an example of tempo and load profile, the execution profile for a “squat” including an eccentric phase duration of 2 seconds, an eccentric-pause phase duration of 0 seconds, a concentric phase duration of 1 second, and a concentric-pause phase duration of 2 seconds may be expressed as:

EP=2 0 1 2

According to embodiments of the present invention, processing the load profile and execution profile information involves determining a single value indicating the total duration of the phases intended to contribute to stress during the execution of the exercise activity.

In the present case the single value may be considered as the actual “total time under tension” (TUT) resulting from the identified phases. In other words, in some embodiments, processing the load profile (LP) information and execution profile (EP) information provides a single value indicating the accumulated time under stress contributed by phases identified by the load profile information during execution of the exercise activity. In the present case, the processing determines a single value as the total time under tension caused by the identified phases as the sum of the products of the values of the load profile and the values of the execution profile. Accordingly, the TUT for each rep in a set may be expressed as:

TUTᵣ=t₁·d₁+t₂·d₂+t₃·d₃+t₄·d₄

or more generally for the set as:

$${TUT}_{R} = \frac{{\sum }_{r = 1}^{{R}_{s}} {t}_{1 , r} \cdot {d}_{1 , r} + {t}_{2 , r} \cdot {d}_{2 , r} + {t}_{3 , r} \cdot {d}_{3 , r} + {t}_{4 , r} \cdot {d}_{4 , r}}{{R}_{s}}$$

In other words, for an exercise activity involving n sets (s), a TUT may be determined for each set as the average TUT per repetition.

Hence, in the example shown in FIG. 3:

LP=1 1 1 0

EP=2 0 1 2

TUT=2·1+0·1+1·1+2·0=3

FIG. 4 includes further examples of TUT derivations for different exercise activities.

In another embodiment of the system, exercise parameters may be determined for each individual repetition rather than using an average of the TUT per repetition. Such an approach may provide additional data in embodiments of the system where performance over time of each set is of interest.

It will also be appreciated that it is possible that exercise activities may be performed in ways to modify the load profile. For example, with a “barbell row” type exercise activity involving manipulation of a weight (wt) to exercise the latissimus dorsi muscle, if the weight is raised and lowered in controlled fashion, then TUT has an impact, in terms of the contribution to stress, during the “up” (that is, the concentric phase) and “down” (that is, the eccentric phase) phase. Furthermore, there is also an impact at the top if the weight is held at the contracted-pause phase, and also at the bottom since the subject is still supporting the weight under stretch in the eccentric-pause phase. However, if the weight is “cheated up”, the TUT will be reduced since the momentum carries the weight up. In this example, this effect occurs because most of the weight is borne by the lower back in “throwing the weight”.

Nevertheless, when the weight is lowered, there is an impact during the “down” phase if the weight is lowered under control and furthermore if the weight can be held or paused at the top there will also be a contribution to stress at this point. On the other hand, if the weight is “cheated” up and down, and not held in the top position, the load profile is effectively 0100 since there is just the load under stretch at the bottom position (that is, the eccentric-pause phase), since the lower back does most of the work at the start to ‘throw’ the weight through the up phase, thus bypassing the muscle targeted by the exercise activity (that is, the latissimus dorsi muscle) and not contributing to stress of the muscle intended to be exercised by the activity. Variations for executing the exercise may be included in the database. For example, the activity database may include “Barbell row 1011”, “Barbell row 1010”, “Barbell row—cheat 0100” or any other means of conveying the exercise performance style and hence the actual load profile.

To explain further, embodiments of the system may provide an option for exercise activities to be performed with a stipulation of continuous tension/load. To clarify the advantage of specifying this variation in style and load profile:
a) exercises activities may have the possibility of reducing or eliminating the load on the muscle at some point of the movement (for example, squats with the knees locked out enable the quads to relax (LP=1110); bicep curls with the arms hanging by the sides enable the biceps to relax, and if the weight is curled to the top such that the forearms are vertical or past vertical, the biceps again are relieved of stress (LP=1010). Hence, the load profile may be specified to take into account these variations:
   a. Curls to arms “hanging” and up to vertical: LP=1010;
   b. Curls to arms “hanging” but short of vertical: LP=1011;
   c. Curls short of full hang or with shoulders slightly flexed as in a “preacher” curl, and stopping at or past vertical: LP=1110; and
   d. Curls short of full hang or with shoulders slightly flexed as in a “preacher” curl, and stopping short of vertical: LP=1111.
b) Similarly, each exercise activity may be performed so as to maintain stress on the working muscle(s):
   a. Squats stopping short of knee lock-out: LP=1111;
   b. Bench press stopping short of elbow lockout: LP=1111;
   c. Tricep lying “EZ extension” stopping short of elbow lockout: LP=1111;
   d. Leg curl stopping short of resting weights on the stack: LP=1111; and
   e. Dumbbell flyes stopping short of full vertical: LP=1111.

Embodiments of the present invention may cater for these variations, and permit the system to determine which variation was used for each execution of an exercise activity.

In some embodiments, the variation may be stipulated by the subject or his/her coach (i.e. by specifying in a recording device which option of performance was used). In other embodiments, the system may detect the option by incorporating, for example, a sensor(s) which conduct a “full range of movement calibration” such that a full range of movement is performed initially with either no weight or a light weight prior to the execution of the actual exercise activity so the system can detect the full eccentric and concentric positions and possibly automatically assign, for each repetition, the appropriate load profile.

For example, prior to executing a barbell curl, the subject may execute a full range repetition. During the execution of the full range repetition the system may detect the 0 and 180 degree positions, then, as the subject executes the barbell curls and the first repetition (“Rep 1”) stops at 75 degrees, the concentric pause of the LP is assigned as “1”, but for the second repetition (“Rep 2”), the arm is extended to a vertical position, in which case the concentric pause of LP is assigned as “0”. In this way, the product of the execution profile and the load profile for the executed exercise activity may be determined as:

EP×LP=[2012]·[1011]=[2012] TUT=5; and Rep 1:

EP×LP=[2022]·[1010]=[2020] and TUT=4. Rep 2:

In other embodiments, the system may estimate the load profile information throughout the range of movement using, for example, a scalar or percentage as opposed to simply assigning a “1” (on load) or “0” (no load). For example, with bicep curls, obtaining the angle of the forearm at the top of the range of movement may allow for the load profile to be estimated as having a value less than 1 (for example, 0.5) to indicate that the bicep is being stressed at 50% during that phase. Furthermore, it is possible that the load profile and duration could be sampled at various points over the range of movement to further improve accuracy.

To further explain load profile, execution profile and impact profile, with a “squat” type exercise activity intended to exercise the quadricep muscles, the “standard” load profile may be “1110” since at the top (that is the concentric-pause phase) of the movement, the load disappears from the quadriceps if the knees are locked. Hence, if a subject executes an execution profile of 1016, that is, a 1 second eccentric phase (that is, during the down movement), no pause at the bottom, a 1 second concentric phase (that is, during up movement) and then rests (with knees locked out) for 6 seconds at the top before the next repetition, according to the present invention the actual TUT is not 8 seconds, but is instead 2 seconds.

Hence if the exercise activity includes 8 repetitions in a set, the set will have a total duration of 58 seconds (noting that the duration is not 64 since on the last rep the subject will effectively ‘rack’ the weight for 6 seconds). However, the effective TUT (that is, the actual working TUT) as determined by the method according to the present invention is 16 seconds.

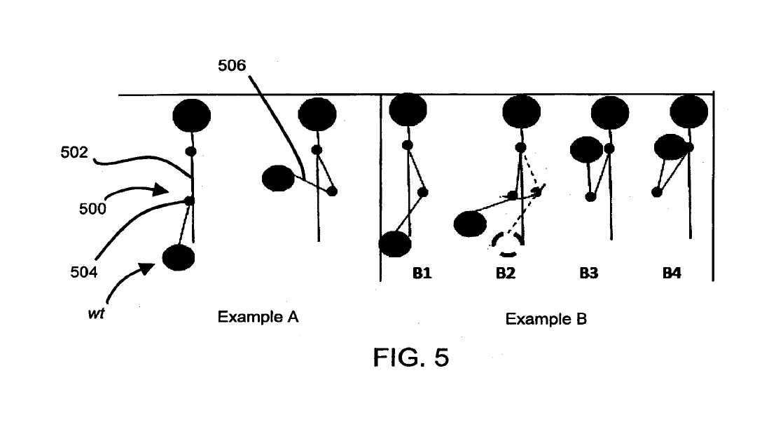

Turning now FIG. 5 there is shown two examples (“Example A” and “Example B”) of a subject's execution of a bicep curl exercise activity. In “Example A” the subject's arm 500 is moved to ensure constant load on the biceps 502, at the start the elbow 504 is slightly forward so the biceps 502 work from the start, and at the top of the movement, the forearm 506 is only just above parallel so the biceps 502 are working hard to contract. On the other hand, in Example B, the elbow 504 is retracted at the start in cheat style meaning that half of the work is already done without actually loading the biceps 502 simply by bringing the elbow forward. (ref position B2) Then at the top of the movement, the elbow 504 is thrust forward and the forearm is vertical (ref position B3), or even in some cases, the weight is allowed to ‘rest’ (ref. position B4). In other words, Example B is a less effective way to perform a curl but is unfortunately used by many subjects to use very heavy weights in the belief that they are making progress, even though this approach is less effective.

In the case of Example B, the execution profile information may be represented as:

EP=1,0,0.5,0

In other words, no “load” at the start or end, and a low “load” during the “up” concentric phase due to ‘throwing’ the weight. In some cases a subject may allow the weight to ‘drop’ in the eccentric phase, as well as bringing the elbows back again so the repetition is not completed properly. In this example, the execution profile information may be represented as:

EP=0.5,0,0.5,0

Having determined the TUT, embodiments of the present invention then process the TUT with the plurality of exercise parameter values for the executed exercise activity to determine one or more assessment parameter values for assessing the executed exercise activity.

In an embodiment, processing the plurality of exercise parameter values to determine one or more assessment parameter values for assessing the executed exercise activity includes determining:
a) a work volume parameter value for the executed exercise activity (W);
b) a work intensity parameter value for the executed exercise activity (Wᵢ);
c) a stress intensity parameter value for the executed exercise activity (Sᵢ); and
d) a hypertrophy factor parameter value for the executed exercise activity (H_{f}).

It is possible that other assessment parameter values may also be determined.

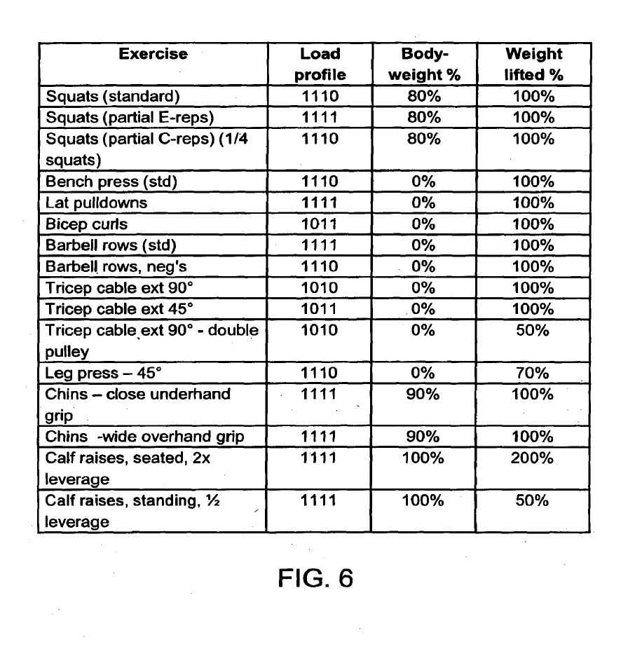

As is shown in FIG. 6, the weight (wt) may include part of the subject's bodyweight. For instance, with squats, about 80% of the body is also lifted (as the lower leg region of each leg is ‘supported’ on the floor and is not lifted, and the upper leg region of each leg is partially supported at the knee joint). With chin-ups, about 90% of the body is lifted (as the hands and forearms are “locked” to the bar and are not lifted). Hence, it is possible that for some exercise activities the weight (wt) may be a proportion of the subject's body weight.

Similarly, with some exercises, the weight lifted (wt) is not the “actual” or apparent weight on the bar. For instance, with a leg press exercise activity on a 45 degree incline, only 60% of the weight loaded is actually transferred to the subject. In other words, the effective weight is a proportion of the total weight manipulated by the subject. There may also be part of the weight of the apparatus to take into account (i.e. a minimum weight such as the weight of the slide or base plate etc.) before the % weight calculation is applied.

For a resistance training type exercise activity involving manipulating a weight (wt) for a number (n) of sets (s) including multiple repetitions (R) over a duration (Tₑ), the assessment parameter values may be determined as follows.

First, a work volume (W) parameter value may be determined as:

$$W = {\sum }_{s = 1}^{n} {W}_{s} = {\sum }_{s = 1}^{n} ( {R}_{s} \cdot {wt}_{s} )$$

where:
Ws: the work volume for sets where s=1 to n
Rs: the number of repetitions in set s
wtₛ: the weight manipulated in set s.

Then, by application of the TUT determined for each set, a stress (S) value may be determined as:

S=R₁·wt₁·TUT₁+R₂·wt₂·TUT₂+ . . . +Rₙ·wtₙ·TUTₙ

which may be further expressed as:

S=Σₛ₌₁ⁿRₛ·wtₛ·TUTₛ

where:
TUTₛ=the TUT value for sets where s=1 to n

Having determined the stress, the stress intensity assessment parameter value may then be determined as:

$${S}_{i} = \frac{S}{{T}_{e}}$$

and the work intensity parameter value as:

$${W}_{i} = \frac{W}{{T}_{e}}$$

The hypertrophy factor assessment parameter may be determined as:

H_{f}=Sᵢ·W

An example of the determination of the assessment parameters is described following to assist the reader in understanding an approach for determining the assessment parameter values.

### Example 1

A subject completed a bench press exercise activity with exercise parameters as listed in table 1 over a total exercise activity duration of 510 seconds.

TABLE 1

| | Primary | Drop Set | Execution |
|---|---|---|---|
| Set | (wt. R) | (wt. R) | Profile (EP) |
| 1 | 10 × 60 | 1010 | |
| 2 | 8 × 70 | 1010 | |
| 3 | 7 × 70 | 1010 | |
| 4 | 9 × 60 | 5 × 50 | 1010 |
| 5 | 10 × 50 | 6 × 40 | 1110 |

The exercise activity was performed as a bench press in which no component of subject's bodyweight was lifted. In this case, the actual weight loaded was the weight lifted (including the bar), and the total work volume was determined as:

W=10×60+8×70+7×70+9×60+5×50+10×50+6×40=3,180 kg-reps

Since the execution profile for sets 1 to 4 of the exercise activity was 1010 and the load profile for bench was 1110 (with elbow lockout), the TUT for sets 1 to 4 was determined as:

TUT=t₁·d₁+t₂·d₂+t₃·d₃+t₄·d₄

TUT=1·1+0·1+1·1+0·1

TUT=2 seconds per repetition

For set 5, the TUT was determined as:

TUT=3 seconds per repetition

The stress (S) was determined as:

$$S = {\sum }_{s = 1}^{n} {R}_{s} \cdot {wt}_{s} \cdot {TUT}_{s}$$

$$S = 10 \times 60 \times 2 + 8 \times 70 \times 2 + 7 \times 70 \times 2 + 9 \times 60 \times 2 + 5 \times 50 \times 2 + 10 \times 50 \times 3 + 6 \times 40 \times 3 \\ S = 7 \text{,} 100 kg \text{-} rep \text{-} secs$$

The work intensity (Wi) was determined as:

$${W}_{i} = \frac{W}{{T}_{e}}$$

$${W}_{i} = \frac{3180}{510}$$

$${W}_{i} = 6.24 kg \text{-} r \text{/} sec$$

### Stress Intensity:

$${S}_{i} = \frac{S}{{T}_{e}}$$

$${S}_{i} = \frac{7100}{510} = 13.92 kg \text{-} r \text{-} s \text{/} s$$

### Hypertrophy Factor:

H_{f}=Sᵢ·W

H_{f}=44,271 units

In this example, the determined assessment parameter values were thus:

| W | S | Wᵢ | Sᵢ | H_{f} |
|---|---|---|---|---|
| 3,180 | 7,100 | 6.24 | 13.92 | 44,271 |

It is to be appreciated that additional assessment parameters may also be determined. For example, embodiments of the present invention may also determine:
Max weight: the maximum of the weight used for any of the sets=Max(₁ⁿ)
Total repetitions: the sum of all the reps of all working sets and drop sets, which in this example=55=Σ₁ⁿ(rₙ+dₙ)
Average weight: the average weight per rep, hence Total Work/Total reps, which in this example=57.8 kg=W/R
Total sets: sum of all working sets (where drop sets are part of a primary set), which in this example=5
Drop sets may also be tallied separately. In this example there are 2 drop sets, hence the work and stress intensities were achieved with the configuration of 5 sets plus 2 drop-sets
Average repetitions per set: total reps/total sets which in this example=55/5=11=R/S (also=W/average weight/rep)

The above example provides assessment values which may permit a subject to readily compare the effectiveness of different exercise activities in terms of their effectiveness on stressing a targeted muscle, as is explained with reference to the below example.

### Example 2

A subject completed a bench press exercise activity with exercise parameters as listed in table 2 over an activity duration of 600 seconds.

TABLE 2

| Set | primary | drop set1 | EP | LP | TUT |
|---|---|---|---|---|---|
| Set 1 | 14 × 50 | | 1010 | 1110 | 2 |
| Set 2 | 10 × 55 | | 1010 | 1110 | 2 |
| Set 3 | 9 × 55 | | 1010 | 1110 | 2 |
| Set 4 | 9 × 55 | 5 × 40 | 1010 | 1110 | 2 |
| Set 5 | 10 × 50 | 4 × 40 | 1110 | 1110 | 3 |

The following assessment parameters were then determined using the process described with reference to Example 1, which for clarity omits other assessment parameters such as the no. of sets, drop sets, max weight, avg weight per rep, total reps, and avg reps per set.

| W | S | Wᵢ | Sᵢ | H_{f} |
|---|---|---|---|---|
| 3,180 | 7,100 | 6.24 | 13.92 | 44,271 |

The assessment parameter values determined in Example 2 were then compared with the corresponding values determined for Example 1 as follows:

| Parameter | Example 1 | Example 2 |
|---|---|---|
| H_{f} | 44,271 | 35,443 |
| Sᵢ | 13.92 | 11.43 |
| Wᵢ | 6.24 | 5.17 |
| W | 3,180 | 3,100 |

The comparison indicated that the exercise activity described in Example 1 was more effective than the exercise activity described in Example 2 in terms of stressing the muscle intended to be exercised by the exercise activity for the goal of hypertrophy or conditioning.

In this manner, the present invention may assist a subject to identify permutations of each exercise activity to achieve maximum effective stress to elicit hypertrophy (or performance response) in each of their given muscles. The output instructions may provide a more ideal target for subjects seeking growth (i.e. H_{f} Sᵢ, Wᵢ) than the traditional methods which are typically pre-set or “prescribed” performance parameters that do not take into account a subject's unique physiology.

Other targets may also be “set” such as maximum work performed or work intensity for the purpose of improving performance in a sport or for increasing calorie expenditure.

Embodiments of the present invention may store assessment parameter values for subsequent analysis to generate instructions for one or more exercise parameters for the future execution of the exercise activity by the subject, such as in a periodised training program. The instructions may provide a subject with plural program selections or options based around their identified ideal performance parameters for each muscle and thus assist the subject in aiming for the new ideal targets via periodised training, to avoid over-training, as explained in further detail as follows.

Embodiments of the present invention may analyse, for example, stored assessment parameter values to identify “peak” exercise activities having, for example, the highest combination of stress intensity and work and set a new target or objective based on these identified “peak” exercise activities. The subject may then work towards a new target by developing strength and work capacity “either side” of the target whilst also allowing recovery between exercise activities. Hence the exercise activities may, for example, “cycle” a load between higher weights and lower volume (and stress) and lighter weight and higher volume, and also cycle periods of higher stress intensity but lower volume (hence lower H_{f}) to stimulate the muscles to develop, whilst reducing the likelihood of physically or psychologically “burn-out”.

The meso-cycles may be determined by calculating variations about the “target” H_{f} exercise activities and varying the loads to avoid over-training as well as provide the necessary stimulus for the subject's muscles to achieve the target. Embodiments of the present invention may thus provide a guide, in the form of a set of instructions, to cycle loads and periodise exercise activities, and may also provide guidelines on how to adjust the exercise activities instinctively. The periodisation may be determined via suitable algorithms.

An embodiment of the present invention may also thereby provide statistics and guidelines for the subject in relation to the exercise parameters over longer periods of time such as micro-cycles, meso-cycles, and macro-cycles. In this manner, the subject will also gather performance and response data for the parameters for any given muscle that have meaning over the longer term, such as total work and analysis of the periodic stress and stress intensity over the longer term.

An example implementation of a method according to an embodiment which generates instructions for one or more exercise parameters for the future execution of the exercise activity by the subject, such as a new performance target or a strategic variation in a periodised training program to reach the new target, is described following.

### Example 3

Exercise Activity: Incline bench
7 sets×15 reps×50 kg, TUT 3 s/rep Rest 86 s/set

Alternative exercise activity instructions recommended by system:
10 sets×6 reps×85 kg, TUT 2 s/rep, Rest 50 s/set

Both of the above exercise activities equate to the same hypertrophy factor H_{f} (that is, Stress intensity×Work volume). However, in this case the system has identified an alternative target for the subject than the traditional “maximum weight” or “max weight for 8 to 12 reps”. By using the present invention, the subject may identify exercise activity combinations which improve the likelihood of the subject achieving the target work capacity, stress intensity and hypertrophy factor that the system determines for him/her.

In order to achieve this new target, the subject may train with varying parameters determined and/or predicted by the system and designed to ultimately develop the aspects of the muscle to achieve that performance, that is, a combination of strength stamina, and work capacity.

Advantageously, specifying “equivalent” targets effectively identifies the preferred parameters for peak performance in an iterative guidance manner.

The varying parameters mentioned above may be determined by the system in a ‘meso-cycle’ program which will stress the muscles around an optimum operating mode as well as providing ‘detraining’ sessions to prevent burn-out. The system may also aim to have the subject achieve peak stimulation not just per workout but also per week, month, and meso-cycle in order to achieve and advance the peak capacity as quickly as possible.

Embodiments of the present invention may analyse recorded exercise activity information to determine one or more exercise activities which involve a slightly increased H_{f}, that also match the subject's capabilities. For example, knowing that the subject can manage an average of 11 reps with 58 kg, provides a guide to the weights possible with other anaerobic repetition ranges.

The historical database of executed exercise activities may categorise exercise activities that have similar effects on the musculature so that exercise activities can be compared as opposed to simply comparing only sessions of the exact same exercise activity. For example, the database may have similar ID coding for flat barbell bench press and flat dumbbell bench press. This approach may enable comparisons of all workout histories of these exercise activities.

### Example 4

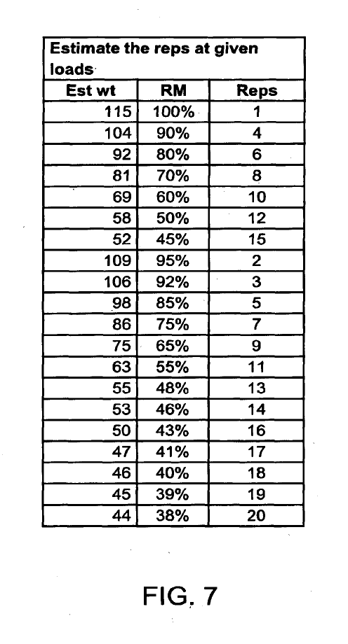

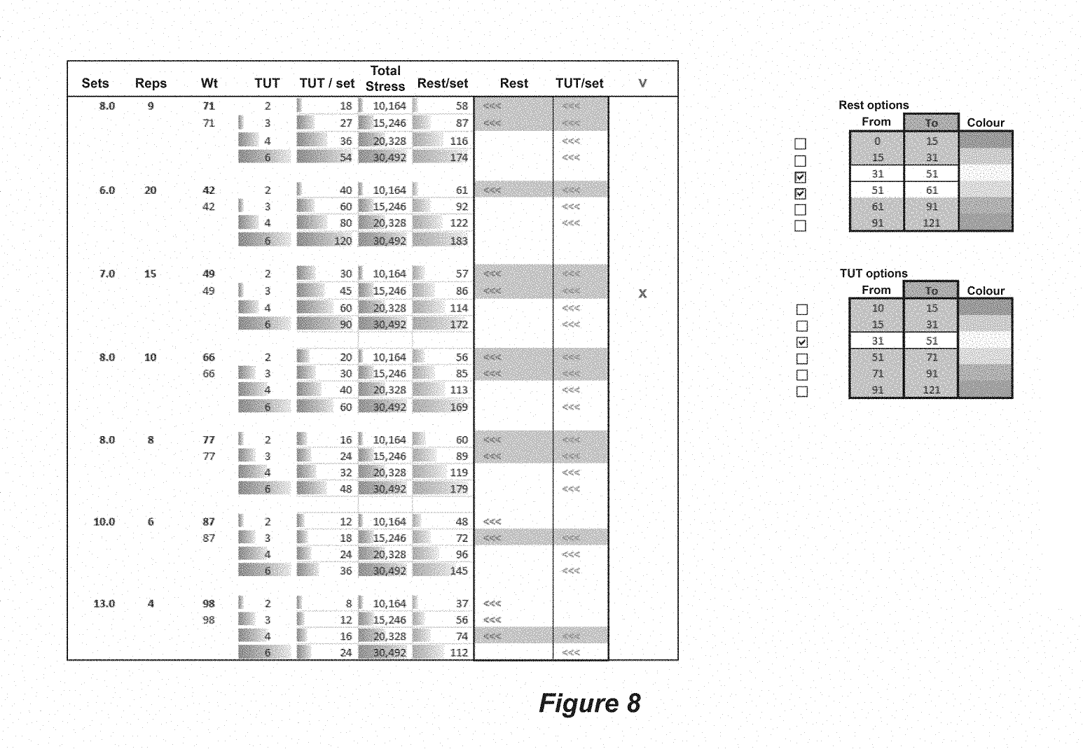

In this example, the average weight for an exercise activity was determined as 58 kg for 12 reps. The system may determine a value expressing the average weight in terms of a proportion, or percentage of 1RM (that is, the subject's one repetition maximum weight for the exercise activity) by indexing, for example, a table in the system database “%1RM vs Reps”. Additional repetition ranges, for different weights, may then be estimated from their typical %1RM values as shown in FIG. 7. This type of table may be calculated for each exercise activity when predicting new target workouts.

Embodiments of the present invention may employ, for example, an existing or standard %1RM vs Reps reference table, and then further refine it over time for each subject in response to the subject's execution of an exercise activity. For example, a standard table might suggest that 8 reps is typically possible with 60%1RM, and by extrapolation that the 70%1RM weight may be determined and, by reverse look-up of the table, 6 reps should be possible. However, for a particular subject in this exercise activity, the subject may demonstrate, on execution of the exercise activity that only 5 reps are possible. In this case, the table may be modified for subject and exercise (over time with the gathering of data), and hence for future predictions when determining new targets.

It is to be understood that refinement of the % RM vs Reps table is not essential. Indeed, it is anticipated that “industry standards” reference tables will be suitable as a guide since, in use, the system calculates what the subject actually achieves, and the subject (or their coach/trainer) learns with experience which targets are reasonable.

In the present case, there are two tables:
Reps (R) vs %1RM (this is a “fixed” reference table)
Reps® vs Weight (W) (this table is calculated for each workout/exercise activity on the basis that for the average weight used vs average reps, the corresponding %1RM for those 4 reps is known and the expected weight for every other %1RM can be determined)

Hence for the table of Reps vs %1RM, weight values can be determined for each Rep value by retrieving the %1RM for the actual reps and pro-rating the values for all other rep values.

That is, for each rep option to be offered, the above tables can be referred to since the predicted reps have been determined based on the actual reps and weight lifted and by relating them to the R v %1RM table. By way of example, the rep options may be as follows:

| | | | | | | | |
|---|---|---|---|---|---|---|---|
| Reps/set | 12 | 20 | 15 | 10 | 8 | 6 | 4 |

For a particular exercise activity (or average of parameters for an exercise activity), an embodiment of the present invention may determine and/or predict the likely weights and repetition ranges. The subject, or the system, may then program an increase of H_{f} or other key parameters, such as:
H_{f} increase (or decrease) %
Sᵢ inc % with W inc % (that is, increase/decrease Si with an increase/decrease W)

It is to be appreciated that the targeted reps/set may not necessarily be limited to the repetitions used in the example.

Alternatively, instead of reps/set, a range of target weights may be used. For example, if the subject's past performance of an exercise activity included 8 reps per set with 50 kg, then targets maybe determined from the predicted 1RM and working on %1RM as follows:

| | | | | | | | |
|---|---|---|---|---|---|---|---|
| wt/set | 80 kg | 70 kg | 60 kg | 55 kg | 50 kg | 45 kg | 40 kg |

Based on the above information, the system may determine a plurality of instructions for executing an exercise activity which meet the training criteria, as follows:
a) Using the various repetitions and the weight/% RM table (ref. FIG. 7), the system determines the weight (wt) for each anaerobic repetition. e.g. 58 kg for 12 reps.
b) Then, for a target work volume (W), the system determines how many repetitions are required for each weight, by indexing the weight/% RM table (ref. FIG. 7).
c) Then, knowing the repetitions possible with each weight, the system determines how many sets will be required to achieve the target work volume for each of a plurality of weight and repetition combinations. For example, if W=3600

$$s = \frac{W}{R \cdot wt}$$

$$s = \frac{3600}{12.58}$$

$$s = 5.2$$

Further examples are shown below.

| Reps/set | 12 | 20 | 15 | 10 | 8 | 6 | 4 |
|---|---|---|---|---|---|---|---|
| Est wt to = W | 58 | 44 | 52 | 69 | 81 | 92 | 104 |
| Reps required | 63 | 83 | 70 | 52 | 45 | 39 | 35 |
| # sets | 5.2 | 4.1 | 4.6 | 5.2 | 5.6 | 6.5 | 9 |

d) From the above determined values, the durations of each set are then calculated using the actual TUT of the execution profile. Note that when determining the initial performance parameters, the load profile was considered against the execution profile to determine an actual load stress. In working backwards to arrive at a target elapsed time for the exercise activity, the predicted duration of each set is required in order to guide the trainee on the required rest between each set. This of course assumes that the trainee performs the reps according to the targeted working TUT—i.e. if the trainee selects an option of weights and reps and a rep TUT of 5 seconds, the system is guiding the trainee such that those 5 seconds are all stressing the muscle (according to the load profile). Hence, if the exercise has a load profile of 1110, and the target TUT is 5 seconds, the reps would be performed as 2120, or 3020 etc. Both of these have a working TUT of 5 seconds. However, if the trainee does not achieve the target or makes a mistake, and for instance performs the reps as 1013 (in other words, “cheating” the movement)—this will show up in the program reports as the trainee will enter their actual TUT as 1013 (particularly if a sensor is used as it will record the phases exactly) and see that the calculated S, Sᵢ, and H_{f} are below target (due to the Working TUT only being 2). They will learn and correct this next time.
e) Various per rep values based on the load profile information for the exercise activity, as follows:

| Est Set | | | | | | | |
|---|---|---|---|---|---|---|---|
| TUT/set | 12 | 20 | 15 | 10 | 8 | 6 | 4 |
| 2 | 24 | 40 | 30 | 20 | 16 | 12 | 8 |
| 3 | 36 | 60 | 45 | 30 | 24 | 18 | 12 |
| 4 | 48 | 80 | 60 | 40 | 32 | 24 | 16 |
| 6 | 72 | 120 | 90 | 60 | 48 | 36 | 24 |

f) The total Work is known as it is a targeted Work volume. The Work per set will depend on the weight and the repetitions from the first table as follows:

| | 12 | 20 | 15 | 10 | 8 | 6 | 4 |
|---|---|---|---|---|---|---|---|
| Work/set | 691 | 876 | 778 | 691 | 645 | 553 | 415 |

g) The Stress per set is then calculated, using the Work per set and the TUT per set, as follows (each row corresponds to a different working TUT):

| | 12 | 20 | 15 | 10 | 8 | 6 | 4 |
|---|---|---|---|---|---|---|---|
| Stress/set | 1383 | 1752 | 1556 | 1383 | 1291 | 1106 | 830 |
| | 2074 | 2627 | 2334 | 2074 | 1936 | 1659 | 1245 |
| | 2766 | 3503 | 3111 | 2766 | 2581 | 2213 | 1659 |
| | 4149 | 5255 | 4667 | 4149 | 3872 | 3319 | 2489 |

h) The total stress for the exercise activity is then determined as Σ₁ⁿ Stressₙ as follows:

| | 12 | 20 | 15 | 20 | 8 | 6 | 4 |
|---|---|---|---|---|---|---|---|
| Total | 7,229 | 7,229 | 7,229 | 7,229 | 7,229 | 7,229 | 7229 |
| stress | 10,844 | 10,844 | 10,844 | 10,844 | 10,844 | 10,844 | 10844 |
| | 14,458 | 14,458 | 14,458 | 14,458 | 14,458 | 14,458 | 14458 |
| | 21,688 | 21,688 | 21,688 | 21,688 | 21,688 | 21,688 | 21688 |

i) The elapsed time Tₑ for the exercise (completion of all sets) is then determined using the targeted Stress Intensity (Stress/Tₑ).

| | 12 | 20 | 15 | 20 | 8 | 6 | 4 |
|---|---|---|---|---|---|---|---|
| Te | 363 | 363 | 363 | 363 | 363 | 363 | 363 |
| | 544 | 544 | 544 | 544 | 544 | 544 | 544 |
| | 725 | 725 | 725 | 725 | 725 | 725 | 725 |
| | 1,088 | 1,088 | 1,088 | 1,088 | 1,088 | 1,088 | 1088 |

j) Hence for all set combinations and total Stress (S) determinations above, the system then determines the Tₑ required to achieve the target S, for all permutations.
k) Finally, since the system has determined the TUT per rep, the number of repetitions per set, and number of sets, it then is able to determine the actual time lifting the weight. Further, since the system knows the total elapsed time Te, and the number of rest periods (#sets less 1), the system calculates the average rest time between sets. (Te−total reps×TUTr))/(#sets−1)
l) For example,

$$( 363 secs - ( 63 reps \times 2 secs ) ) / ( 5 - 1 )$$

$$( using the top left figures from the tables ) . \\ \begin{matrix}= ( 363 - 126 ) / 4 ) \\ = 237 / 4 \\ = 59 seconds rest average per set\end{matrix}$$

Further examples of rest period determinations are shown below for corresponding reps per set (columns) and working TUT (rows):

| | 12 | 20 | 15 | 20 | 8 | 6 | 4 |
|---|---|---|---|---|---|---|---|
| Rest/set | 59 | 66 | 56 | 65 | 55 | 47 | 37 |
| | 89 | 99 | 84 | 97 | 82 | 71 | 55 |
| | 119 | 132 | 112 | 129 | 109 | 95 | 73 |
| | 178 | 198 | 167 | 194 | 164 | 142 | 110 |

m) The rest period is then calculated for each weight, rep, and TUT variation. All of these permutations represent the same target H_{f}, or Sᵢ and W combinations.

Having calculated the parameters outlined above, embodiments of the present invention then output the results in the form of a set of instructions for executing the exercise.

FIG. 8 shows one example of an output 800 for display. The illustrated output 800 provides a user-friendly presentation of the weight/rep/TUT and rest combinations to achieve a target H_{f}, or W and Sᵢ.

Another element shown in the output shown in FIG. 8 is a preference tool of rest and TUT options to help the subject select an exercise parameter combination as may be done for example via intuitive periodisation or for gaining more data about a muscles working capacity in order to better identify the optimum training parameters. For example, the subject may have trained heavier last workout (with less reps and therefore lower TUT per set) and therefore may wish to choose a workout with higher reps and longer TUT per set (as part of the meso-cycle and adaptation process). They may also have trained with longer rests and want to shorten the rests to increase the intensity. In the illustrated output, the subject may therefore “tick”, in this example, the “Rest=31 to 51” seconds option to highlight all exercise activities matching that criteria (as identified the “rest” column with “<<<<”)

The subject may then “tick”, in this example, the rest options “31-51” and “51-61” to identify rest periods 31 to 61 seconds, and highlight those workouts (as identified in the TUT column with “<<<<” or by other highlighting means).

Further, where in the event that both criteria are met, the system highlights the indicators in both columns.

Some exercise activities may be possible for the subject and others may be too challenging. However, through using the system it is anticipated that the subject will learn their limitations and will intuitively select the combinations more likely to be achievable. In this way, the system “adapts” to the subject and finds their optimum training style for each muscle/exercise. This applies even over time of the subject's performance changes with age or other factors.

### Filtering the Selection

In an embodiment of the invention, various options for the next workout targets may be filtered for display to assist with the selection process. For example:
a) A filter may be provided to filter the desired repetition ranges. For example, 4 to 8; 9 to 12; 13 to 15; 16 to 25;
b) A filter may be provided to filer rest period options. For example, 15 to 30 seconds; 31 to 60 seconds; 61 to 90 seconds; 91 and above seconds
c) A filter may be provided to filter may be TUT per set options. For example, 5 to 16 seconds; 17 to 30 seconds; 31 to 59 seconds; 60 seconds and above
d) Or other combinations.

### Drop Sets

An embodiment of the present invention may also include guidance for using drop-sets in the target workouts.

This may be achieved as follows:
a) Calculating target options for various repetition schemes as identified previously;
b) Selecting target workout parameters for an exercise activity, for example:

bench press; 7 sets×15 reps×50 kg, TUT 3 s/rep Rest 86 s/set;
c) Selecting an option to execute the exercise activity as a “drop set”;
d) In an embodiment, the drop sets are determined by:
   a. Revise the “primary” sets as P′=P sets×0.8;
   b. Weights and reps for P′ remain unaltered of course as these are known from history to be the subject's capabilities—however the Work is now reduced and needs to be restored by the drop sets;
   c. Determine the Work from the P′×weights×reps=W−W′;
   d. Determine the number of drop sets as P′/2 (hence the total sets are increased by 20% of the original prescription—that is, if the initial set prescription P was 5 sets, total sets will now be 6 (120% based on 80%+50% of 80%) such that 4 sets are now the primary (40% of 5) and 2 sets are drop sets (50% of 4);
   e. Determine the weight for the drop sets based on 80% of the weight of the primary set. For example, if the primary set is 50 kg as in this example, the drop set will be 40 kg;
   f. Determine the Work in the drop-sets required to bring the total work back to the original Work;—W−(P′×wt×Reps×Sets×80%);
   g. Determine the Work intensity, knowing the TUT for all sets and the fact that the drop set is executed theoretically as zero seconds from the primary set;
   h. Note that Te (elapsed time for all 6 sets) will be the same as the original Te since Work is now the same and intensities are required to be the same;
   i. Determine the duration of the drop sets (knowing the number of reps and the TUT); and
   j. Determine the revised rest between the primary sets, knowing the set durations, the zero rest from primary to drop sets, and the total exercise duration Te.

In another embodiment a system in accordance with the present invention may provide an option as above known as a “standard” drop set, or allow selection of a “customised” drop set criteria. A customised drop set criteria may enable the subject or coach to pre-set the system with choices of various combinations such as:
Primary sets P′=1, 2, 3, 4;
Or primary sets P′=80% P, 60% P, 50% P;
Drop set weight wt′=80% wt, 60% wt, 50% wt; and
The calculations in these cases are performed in similar fashion as per the explanation for the current “standard” drop set calculations.

### Real-Time Guidance

In an embodiment of the invention, the system may perform the calculations for the current exercise activity in real-time and provide feedback to the subject in real-time, or near real-time, on their progress to meeting their target.

For instance the system might indicate that based on the Work and times thus far in the exercise activity and that only one set remains, that if the subject starts the set in 20 seconds and performs the desired reps in the specified form, the subject will achieve their target.

Furthermore, embodiment of the present invention display a total H_{f} score that will be achieved if the subject starts the last set in the within a particular duration (for example, 20 seconds), and then updates this score periodically (for example, every 5 seconds) and displays the updated score against the previous best score.

### Phase Timing Guidance (EP), Audible

In another embodiment of the invention, the system may provide an alert for each phase of the exercise (for example, for each phase of the targeted execution profile for each rep) to assist the subject with the timing of the phases during the exercise activity. For instance, the system may provide an audible tome or “beep” at the start of each repetition phase, in other words, the eccentric phase, the eccentric pause phase, the concentric-phase, or the concentric-pause phase to indicate the tempo of an exercise activity. In this way, if the subject, for example, is in the eccentric phase and commences the concentric pause phase before the next “beep”, they know they are going too fast, etc.

In view of the above, it will be appreciated that the present invention provides a plurality of exercise activity instructions of varying weight, reps, TUT, rest that all match a targeted Hf or Si and W. Furthermore, the present invention is expected to assist a subject identify exercise activities which match their preference when planning their next workout. Preferably, this process is accomplished in one operation for all exercises activities that the subject plans for the next workout. The trainee can then select the preferred parameters for each exercise by “ticking” the workout that matches their preferred characteristics.

The system then downloads this workout data to a printable area as well as a field that can be exported to, for example, SMS or other e-media output or displayed.

It should be appreciated that the present invention can be implemented in numerous ways, including as a process, an apparatus, a system, or a computer readable medium such as a computer readable storage medium or a computer network wherein program instructions are sent over wireless, optical, or electronic communication links. It should be noted that the order of the steps of disclosed processes may be altered within the scope of the invention.

Details concerning computers, computer networking, software programming, telecommunications and the like may at times not be specifically illustrated as such were not considered necessary to obtain a complete understanding nor to limit a person skilled in the art in performing the invention, are considered present nevertheless and as such are considered to be within the skills of persons of ordinary skill in the art.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Although the foregoing invention has been described in some detail for purposes of clarity of understanding, it will be apparent that certain changes and modifications may be practiced within the scope of the appended claims. It should be noted that there are many alternative ways of implementing both the process and apparatus of the present invention. Accordingly, the present embodiments are to be considered as illustrative and not restrictive, and the invention is not to be limited to the details given herein, but may be modified within the scope and equivalents of the appended claims.

Throughout this specification and the claims that follow unless the context requires otherwise, the words ‘comprise’ and ‘include’ and variations such as ‘comprising’ and ‘including’ will be understood to imply the inclusion of a stated integer or group of integers but not the exclusion of any other integer or group of integers.

The reference to any background or prior art in this specification is not, and should not be taken as, an acknowledgment or any form of suggestion that such background or prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that various modifications can be made without departing from the principles of the invention. Therefore, the invention should be understood to include all such modifications within its scope.

Although a preferred embodiment of the method and system of the present invention has been described in the foregoing detailed description, it will be understood that the invention is not limited to the embodiment disclosed, but is capable of numerous rearrangements, modifications and substitutions without departing from the scope of the invention as set forth and defined by the following claims.

1. A method of analysing a resistance exercise activity executed by a subject, the method including:
receiving a plurality of exercise parameter values for the executed exercise activity into a processing device, the plurality of exercise parameter values including information representing an execution profile (EP) for the executed exercise activity;
accessing a store of information to retrieve information representing a load profile (LP) for the executed exercise activity; and
processing the plurality of exercise parameter values and the information representing the load profile to determine one or more assessment parameter values for assessing the executed exercise activity.

2. A method according to claim 1 wherein the exercise activity executed by the subject includes a resistance training exercise activity involving one or more weights (wt), one or more sets (s), each set including one or more repetitions (R), and a total activity time (Tₑ), and wherein the plurality of exercise parameter values include:
a. a set parameter value (n) representing the number of the one or more sets (s) for the resistance training activity;
b. a weight parameter value (wtₛ) for the weight associated with a set s where s=1 to n;
c. a repetition parameter value (Rₛ) representing the number of one or more repetitions in a set s where s=1 to n; and
d. a total activity time parameter value (Tₑ) being the time elapsed from the first repetition of the first set (s=1) to the final repetition of the final set (s=n) of the exercise activity.

3. A method according to claim 2 wherein the execution profile information and the load profile information include corresponding sequences of values, wherein each value is associated with a different exercise phase of the exercise activity for a muscle of the subject intended to perform work during the exercise activity, and wherein the values associated with different exercise phases of the exercise activity include values associated with:
a. an eccentric phase;
b. an eccentric-pause phase;
c. an concentric phase; and
d. a concentric-pause phase.

4. A method according to claim 2 further including accessing an exercise activity database to retrieve a parameter indicating a proportion of the subject's bodyweight contributing to a work performed by the exercise activity, and determining each weight parameter value (wtₛ) based at least in part on the parameter indicating the proportion of the subject's bodyweight, the subject's bodyweight contributing to the work performed by the exercise activity, and an exercise load.

5. A method according to claim 3 wherein the load profile (LP) information identifies the exercise phases intended to contribute to work during execution of the exercise activity, and wherein the load profile (LP) information is expressed as a sequence values, the sequence including a value (d₁) identifying an eccentric phase contribution, a value (d₂) indicating an eccentric-pause phase contribution, a value (d₃) indicating a concentric phase contribution, and a value (d₄) indicating an concentric-pause phase contribution.

6. A method according to claim 5 wherein the load profile (LP) information is expressed as the sequence [d₁, d₂, d₃, d₄], and wherein each value in the sequence is a binary digit having a first value for indicating that the respective exercise phase is intended to contribute to work, and a second value for indicating that the respective exercise phase is not intended to contribute to work.

7. A method according to claim 5 wherein each value in the sequence of values includes a scalar value indicating a proportion of a load utilised in working a muscle during the respective exercise phase.

8. A method according to claim 3 wherein the execution profile (EP) information includes a value (t₁) indicating the duration of the eccentric phase during the execution of the exercise activity, a value (t₂) indicating the duration of the eccentric-pause phase during the execution of the exercise activity, a value (t₃) indicating the duration of the concentric phase during the execution of the exercise activity and a value (t₄) indicating the duration of the concentric-pause phase during the execution of the exercise activity.

9. A method according to claim 8, wherein the execution profile information is sensed during execution of the exercise activity.

10. A method according to claim 8 wherein processing the plurality of exercise parameter values and the activity information to determine one or more assessment parameter values for assessing the executed exercise activity includes determining a value of total time under tension (TUT) for each repetition:
TUT_{R}=t_{1,r}·d_{1,r}+t_{2,r}·d_{2,r}+t_{3,r}·d_{3,r}+t_{4,r}·d_{4,r}.

11. A method according to claim 10 wherein for each set (s) a single value of time under tension (TUT) is determined as an average value of time under tension for the repetitions of a set as:
$${TUT}_{R} = \frac{{\sum }_{r = 1}^{{R}_{s}} {t}_{1 , r} \cdot {d}_{1 , r} + {t}_{2 , r} \cdot {d}_{2 , r} + {t}_{3 , r} \cdot {d}_{3 , r} + {t}_{4 , r} \cdot {d}_{4 , r}}{{R}_{s}}$$

12. A method according to claim 2 wherein the one or more assessment parameter values for assessing the executed exercise activity include at least one of:
a. a work volume parameter value for the executed exercise activity (W);
b. a work intensity parameter value for the executed exercise activity (Wᵢ);
c. a stress intensity parameter value for the executed exercise activity (Sᵢ); and
d. a hypertrophy factor parameter value for the executed exercise activity (H_{f}).

13. A method according to claim 12 wherein the work volume parameter value (W) is determined as:
W=Σₛ₌₁ⁿWₛ=Σₛ₌₁ⁿ(Rₛ·wtₛ)
where:
n is the number of sets in the exercise activity;
Rₛ is the number of repetitions in set s, where s=1 to n; and
wtₛ is the weight associated with each set where s=1 to n.

14. A method according to claim 13 wherein the work intensity parameter value (Wᵢ) is determined as:
$${W}_{i} = \frac{W}{{T}_{e}}$$

15. A method according to claim 12 wherein for each set (s) a single value of time under tension (TUT) is determined as an average value of time under tension for the repetitions of a set as:
$${TUT}_{R} = \frac{{\sum }_{r = 1}^{{R}_{s}} {t}_{1 , r} \cdot {d}_{1 , r} + {t}_{2 , r} \cdot {d}_{2 , r} + {t}_{3 , r} \cdot {d}_{3 , r} + {t}_{4 , r} \cdot {d}_{4 , r}}{{R}_{s}}$$

16. A method according claim 15 wherein the stress intensity parameter value (Sᵢ) is determined as:
$${S}_{i} = \frac{{\sum }_{s = 1}^{n} {R}_{s} \cdot {wt}_{s} \cdot {TUT}_{R}}{{T}_{e}}$$

17. A method according to claim 16 wherein the hypertrophy factor parameter value (H_{f}) is determined as:
H_{f}=Sᵢ·W

18. A method of analysing a resistance exercise activity executed by a subject, the method including:
receiving a plurality of exercise parameter values for the executed exercise activity into a processing device, the plurality of exercise parameter values including information representing an execution profile (EP) for the executed exercise activity;
accessing a store of information to retrieve information representing a load profile (LP) for the executed exercise activity; and
processing the plurality of exercise parameter values and the information representing the load profile to determine one or more assessment parameter values for assessing the executed exercise activity;
wherein the execution profile information and the load profile information include corresponding sequences of values associated with a different exercise phase of the exercise activity for a muscle of the subject intended to perform work during the exercise activity, such that the values associated with different exercise phases of the exercise activity include values associated with:
an eccentric phase;
an eccentric-pause phase;
an concentric phase; and
a concentric-pause phase;
and wherein the load profile (LP) information identifies the exercise phases intended to contribute to work during execution of the exercise activity, and the execution profile (EP) information identifies the duration of each exercise phase which contributed to work during execution of the exercise activity.

19. A computer readable media including computer program instructions which are executable by a processor to implement a method according to claim 1.

20. A system for analysing a resistance exercise activity executed by a subject, the system including:
a processing unit programmed with a set of programmed instructions in the form of a computer software program;
a store of information representing a load profile (LP) for the executed activity; and
receiving means for receiving a plurality of exercise parameter values for the executed exercise activity into the processing unit, the plurality of exercise parameter values including information representing an execution profile (EP) for the executed exercise activity;
wherein the processing unit retrieves the information representing a load profile (LP) for the executed exercise activity and processes the plurality of exercise parameter values of the execution profile information and the information representing the load profile to determine one or more assessment parameter values for assessing the executed exercise activity.

21. A system according to claim 20 where the exercise activity executed by the subject includes a resistance training exercise activity involving one or more weights (wt), one or more of sets (s), each set including one or more repetitions (R), and a total activity time (Tₑ), and wherein the plurality of exercise parameter values includes:
a. a set parameter value (n) representing the number of the one or more sets (s) for the resistance training activity;
b. a weight parameter value (wtₛ) for the weight associated with a set s where s=1 to n;
c. a repetition parameter value (Rₛ) representing the number of one or more repetitions in a set s where s=1 to n; and
d. a total activity time parameter value (Tₑ).

22. A system according to claim 20 wherein the execution profile information and the load profile information include corresponding sequences of values, wherein each value is associated with a different exercise phase of the exercise activity, and wherein the values associated with different exercise phases of the exercise activity include values associated with:
a. an eccentric phase;
b. an eccentric-pause phase;
c. a concentric phase; and
d. a concentric-pause phase.

23. A system according to claim 20 further including an exercise activity database which is accessible by the processing unit to:
retrieve a parameter indicating a proportion of the subject's bodyweight contributing to a work performed by the exercise activity; and
determine each weight parameter value (wtₛ) based at least in part on the parameter indicating the proportion of the subject's bodyweight, the subject's bodyweight contributing to the work performed by the exercise activity, and an exercise load.

24. A system according to claim 22 wherein the load profile (LP) information identifies the exercise phases intended to contribute to work during execution of the exercise activity and wherein the load profile (LP) information is expressed as a sequence of values, the sequence including a value (d₁) identifying an eccentric phase contribution, a value (d₂) indicating an eccentric-pause phase contribution, a value (d₃) indicating a concentric phase contribution, and a value (d₄) indicating an concentric-pause phase contribution.

25. A system according to claim 23 wherein the load profile (LP) information is expressed as the sequence [d₁, d₂, d₃, d₄], and wherein each value in the sequence is a binary digit having a first value indicating that the respective exercise phase is intended to contribute to work, and a second value indicating that the respective exercise phase is not intended to contribute to work.

26. A system according to claim 22, wherein the execution profile (EP) information includes a value (t₁) indicating the duration of the eccentric phase during the execution of the exercise, a value (t₂) indicating the duration of the eccentric-pause phase during the execution of the exercise, a value (t₃) indicating the duration of the concentric phase during the execution of the exercise and a value (t₄) indicating the duration of the concentric-pause phase during the execution of the exercise.

27. A system according to claim 26 wherein processing the plurality of exercise parameters values and the activity information to determine one or more assessment parameter values for assessing the executed exercise activity includes determining a value of total time under tension (TUT) for each repetition:
TUT_{R}=t_{1,r}·d_{1,r}+t_{2,r}·d_{2,r}+t_{3,r}·d_{3,r}+t_{4,r}·d_{4,r}

28. A system, according to claim 27 wherein for each set (s) the single time under tension value (TUT) is determined as an average time under tension value for the repetitions of a set as:
$${TUT}_{R} = \frac{{\sum }_{r = 1}^{{R}_{s}} {t}_{1 , r} \cdot {d}_{1 , r} + {t}_{2 , r} \cdot {d}_{2 , r} + {t}_{3 , r} \cdot {d}_{3 , r} + {t}_{4 , r} \cdot {d}_{4 , r}}{{R}_{s}}$$

29. A system according to claim 20 wherein the one or more assessment parameter values for assessing the executed exercise activity include:
a. a work volume parameter value for the executed exercise activity (W);
b. a work intensity parameter value for the executed exercise activity (Wᵢ);
c. a stress intensity parameter value for the executed exercise activity (Sᵢ); and
d. a hypertrophy factor parameter value for the executed exercise activity (H_{f}).

30. A system according to claim 29 wherein the work volume parameter value (W) is determined as:
$$W = {\sum }_{s = 1}^{n} {W}_{s} = {\sum }_{s = 1}^{n} ( {R}_{s} \cdot {wt}_{s} )$$
where:
n is the number of sets in the exercise activity;
Rₛ is the number of reps in set s, where s=1 to n; and
wtₛ is the weight associated with sets where s=1 to n.

31. A system according to claim 30 wherein the work intensity parameter value (Wᵢ) is determined as:
$${TUT}_{R} = \frac{{\sum }_{r = 1}^{{R}_{s}} {t}_{1 , r} \cdot {d}_{1 , r} + {t}_{2 , r} \cdot {d}_{2 , r} + {t}_{3 , r} \cdot {d}_{3 , r} + {t}_{4 , r} \cdot {d}_{4 , r}}{{R}_{s}}$$

32. A method according to claim 31 wherein for each set (s) a single value of time under tension (TUT) is determined as an average value of time under tension for the repetitions of a set as:
$${W}_{i} = \frac{W}{{T}_{e}}$$

33. A system according to claim 32, wherein the stress intensity parameter value (Sᵢ) is determined as:
$${S}_{i} = \frac{{\sum }_{s = 1}^{n} {R}_{s} \cdot {wt}_{s} \cdot {TUT}_{R}}{{T}_{e}}$$

34. A system according to claim 29 wherein the hypertrophy factor parameter value (H_{f}) is determined as:
H_{f}=Sᵢ·W
